(19) United States
(12) Patent Application Publication
(10) Pub. No.: US 2026/0007365 A1
(43) Pub. Date: Jan. 8, 2026

(54) NON-INVASIVE SENSOR APPARATUS AND METHOD FOR ASSESSING CARDIAC PERFORMANCE OF THE FEET

(71) Applicant: ACCU-THERM SYSTEMS, INC., Ann Arbor, MI (US)

(72) Inventors: Raj Riswadkar, Clayton, MO (US); Pedro Martinez-Clark; Joseph R. Korotko, Livonia, MI (US); Dean Dubois, Clearwater, FL (US)

(21) Appl. No.: 19/130,715

(22) PCT Filed: Nov. 16, 2023
(86) PCT No.: PCT/US2023/079987

§ 371 Date: May 16, 2025

Related U.S. Application Data
(60) Provisional application No. 63/425,792, filed on Nov. 16, 2022.

(51) Int. Cl. A61B 5/00; A61B 5/01; A61B 5/02; A61B 5/029

(52) U.S. Cl. CPC A61B 5/702; A61B 5/01; A61B 5/02007; A61B 5/029; A61B 5/4842; A61B 5/7278

(57) ABSTRACT

A device for measuring a temperature profile of a patient's extremity include a support structure having a plurality of openings therein, a frame delineating each opening of the plurality of openings, a sensor received in each frame, and a plurality of rods connecting each sensor to each frame. A system for monitoring a temperature profile of a patient's extremity and a method of monitoring a temperature profile of a patient's extremity are also disclosed.

### CROSS-REFERENCE TO RELATED APPLICATIONS

This application is a 371 application of International Application No. PCT/US2023/079987, filed Nov. 16, 2023, which claims priority to U.S. Provisional Application Ser. No. 63/425,792 filed on Nov. 16, 2022, which is hereby incorporated herein by reference in its entirety.

### BACKGROUND

A temperature profile, or isogram, of a patient's extremity (such as a hand or foot) is an indicator of the cardiac/vascular status of the patient. More particularly, the temperature profile is indicative of blood perfusion through the extremity. For instance, the temperature profile could be used to monitor for critical limb ischemia (CLI), assess vascularization or revascularization (for example, to guide interventional procedures and to evaluate their efficacy), diagnose the cause of claudication, and measure cardiac hemodynamics such as systemic vascular resistance (SVR) and cardiac output (CO). The temperature profile can be created by measuring the skin temperature at various points on the patient's extremity.

However, there are certain drawbacks associated with traditional methods of monitoring a patient's cardiac/vascular status by skin temperature. Existing technologies that measure blood perfusion including skin perfusion pressure (SPP), duplex ultrasound (DUS), and transcutaneous oxygen monitoring (TOM) suffer from one or more disadvantages. SPP only provides perfusion data at the skin dermis level, and is unreliable in patients with edema. SPP also requires the use of a pressure cuff. DUS typically can only measure blood flow in large vessels (>1.5 mm). TOM requires the patient to be placed on hyperbaric oxygen, which is incompatible with certain settings.

Accordingly, there is a need for reliable, noninvasive, real-time measurement of blood perfusion on in a patient's extremity.

### SUMMARY OF THE INVENTION

A device for measuring a temperature profile of a patient's extremity according to an exemplary embodiment of this disclosure, among other possible things include a support structure having a plurality of openings therein, a frame delineating each opening of the plurality of openings, a sensor received in each frame, and a plurality of rods connecting each sensor to each frame.

In a further example of the foregoing, at least one of the plurality of rods is adjustable in length.

In a further example of any of the foregoing, at least one of the plurality of rods includes a telescoping mechanism.

In a further example of any of the foregoing, the frame is rigid or semi-rigid.

In a further example of any of the foregoing, the frame is at least as rigid as the support structure.

In a further example of any of the foregoing, the support structure is flexible.

In a further example of any of the foregoing, the device includes at least 7 sensors.

A system for monitoring a temperature profile of a patient's extremity according to an exemplary embodiment of this disclosure, among other possible things include a device for measuring a temperature profile of a patient's extremity. The device includes a support structure having a plurality of openings therein, a frame delineating each opening of the plurality of openings, a sensor received in each frame, and a plurality of rods connecting each sensor to each frame. A controller is operable to receive temperature data from the sensors. A computing device is configured to analyze the temperature data from the sensors.

In a further example of the foregoing, the controller is configured to send a request to the sensors to make a temperature measurement.

In a further example of any of the foregoing, the computing device is configured to determine a cardiac profile of the patient based on analysis of the temperature data from the sensors.

In a further example of any of the foregoing, the computing device is configured to determine the patient's systemic vascular resistance based on analysis of the temperature data from the sensors.

In a further example of any of the foregoing, the computing device is configured to determine the patient's cardiac output based on analysis of the temperature data from the sensors.

In a further example of any of the foregoing, the computing device is configured to compare the cardiac profile of the patient to a predetermined normal cardiac profile.

In a further example of any of the foregoing, the computing device is configured to determine a change in the patient's cardiac profile over time based on analysis of the temperature data from the sensors.

In a further example of any of the foregoing, the system also includes a user interface configured to receive an input from a user and communicate the input to the controller.

A method of monitoring a temperature profile of a patient's extremity according to an exemplary embodiment of this disclosure, among other possible things include adjusting a plurality of temperature sensors onto the patient's extremity to correspond with a plurality of anatomical areas of interest, determining a temperature at each of the anatomical areas of interest, and using the plurality of temperatures to determine a cardiac profile of the patient.

In a further example of the foregoing, the anatomical areas of interest include pedal angiosomes.

In a further example of any of the foregoing, the anatomical areas of interest include points of arterial superficiality.

In a further example of any of the foregoing, the method also includes comparing the cardiac profile of the patient to a predetermine normal cardiac profile.

In a further example of any of the foregoing, the cardiac profile includes at least one of cardiac output and systemic vascular resistance.

### BRIEF DESCRIPTION OF THE DRAWINGS

FIG. 1 schematically shows an example device for measuring the skin temperature of a patient's extremity.

FIG. 2 schematically shows a detail view of a sensor of the device of FIG. 1.

FIG. 3 schematically illustrates a system for monitoring the temperature profile of a patient's extremity.

### DETAILED DESCRIPTION

A temperature profile, or isogram, of a patient's extremity can be used to indicate the cardiac/vascular status of the patient. The temperature of the patient's skin at the extremity relates to blood flow throughout the extremity (known as perfusion), which in turn relates to various critical hemodynamic metrics which are indicative of the patient's overall cardiac/vascular health. Thus information about the temperature profile can be used for detecting disease, for prevention of disease by monitoring changes or progression in cardiac/vascular health, of for assessing the outcome of an interventional procedure. For instance, the temperature profile could be used to monitor for critical limb ischemia (CLI), assess vascularization or revascularization (for example, to guide interventional procedures and to evaluate their efficacy), diagnose the cause of claudication, and measure/monitor cardiac hemodynamics such as systemic vascular resistance (SVR) and cardiac output (CO). The extremity can be a hand or foot, for example.

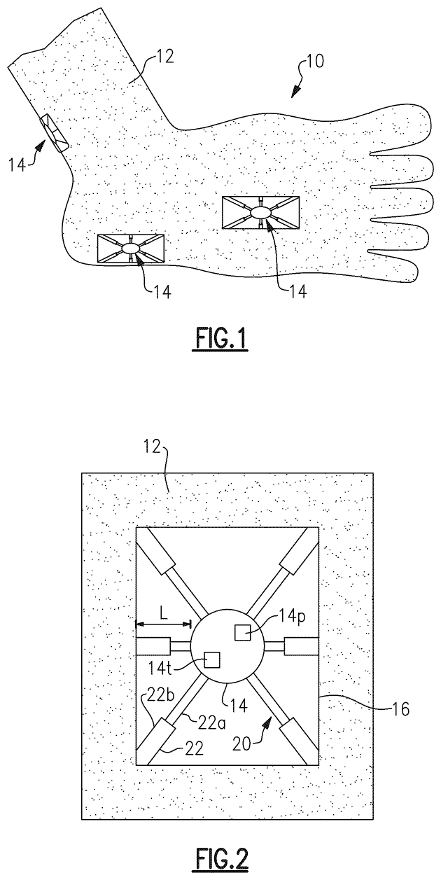

FIG. 1 schematically illustrates a device 10 for measuring the skin temperature of a patient's extremity. The device 10 generally includes a support structure 12 with a plurality of sensors 14 disposed on the support structure 12. In the example of FIG. 1, the support structure is in the shape of a sock but in other examples could take other forms as discussed above.

The support structure 12 can be made from a flexible material, such as thermoplastic elastomer, silicone, or nylon. The material can be a fabric in some examples. For instance, the support structure 12 can be in the form of a toe sock that can be worn on the patient's foot, or a glove that can be worn on a patient's hand.

The sensors 14 are operable to measure the temperature of the patient's skin and communicate the temperature data to a controller as is discussed in more detail below. Any known temperature sensor could be used. Where the extremity is a foot, the sensors 14 can be on the dorsal side, the planar side, or both sides of the patient's foot. Likewise the sensors 14 could be arranged on one or both sides of the patient's hand.

As discussed in more detail below, the sensors 14 could include an integrated transmitter 14t or could have a separate transmitter 14t.

The sensors 14 are arranged on the support structure 12 such that when the support structure 12 is placed on a patient's extremity, the sensors 14 contact the patient's skin. In some examples, the sensors 14 include a pressure sensing element 14p which is configured to sense whether the pressure is in contact with the patient's skin. The pressure sensing element 14b could by any known pressure sensor, and could be integrated in the sensors 14 or could be a separate sensing element.

Moreover, the location of the sensors 14 on the support structure 12 can correspond to certain anatomical areas of interest. For instance, where the extremity is a foot, the sensors 14 could be arranged to correspond to the location of pedal angiosomes or points of arterial superficiality, which are known in the art. For example, sensors 14 can be arranged to correspond to a patient's Medial and Lateral Planter, Post Tibial, Dorsal Pedis, or the Accuate arteries, among others. In some particular examples, the device 10 includes at least 7 sensors. In further examples, the device 10 includes up to 20 sensors 14.

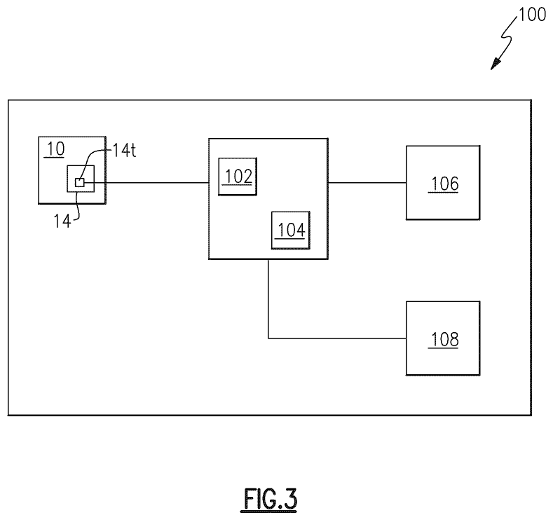

FIG. 2 shows a detail view of a sensor 14. As shown, each sensor 14 is arranged in a frame 16. The frame 16 delineates an opening 18 in the support structure 12 that receives the sensor 14. The frame 16 can be rigid or semi-rigid, in some examples. In most cases, the frame 16 will be as rigid or more rigid than the support structure 12. Though the frame 16 is shown in FIG. 2 as having a generally rectangular shape, it should be understood that other shapes could also be used.

A plurality of rods 20 connect the sensor 14 to the frame 16. One or more of the rods 20 is adjustable. For instance, the rods 20 can have a telescoping mechanism 22 that enables the length L of the rod 20 to be adjusted. The telescoping mechanism 22 includes an inner rod 22a and outer rod 22b that can be extended with respect to one another or compressed by storing the inner rod 22a inside the outer rod 22b, as is well known in the art for telescoping mechanisms. However, it should be understood that other known adjustment mechanisms for the length of a rod could be used. In this way, the frame 16/rods 20 support the sensors 20 in the support structure 12 in an adjustable manner. The adjustment is in the plane of the opening 18, e.g., generally in two dimensions. In the example of FIG. 2, there are six rods 20, but more or less rods 20 could be used. The rods 20 may also include a lock feature to lock the rod 20 in a desired length L.

Though as discussed above the sensors 14 are arranged on the support structure 12 such that they correspond to certain anatomical areas of interest, there can be anatomical differences such that the particular location of the anatomical features vary slightly from patient to patient. Moreover, the size of the patient's foot/hand can affect the relative positioning of the anatomical areas of interest. Typically, the variation is on the order of millimeters to centimeters. Accordingly, the adjustable rods 20 allow a user, such as a medical provider, to adjust the precise position of the sensors 14 within the opening 18 of the support structure 12 according to the particular patient's anatomy. This in turn improves the quality of the sensor 14 readings and improves the accuracy of the analysis of the data collected by the sensor, which is discussed in more detail below.

FIG. 3 schematically illustrates a system 100 for monitoring the temperature profile of a patient's extremity. The system 100 includes the device 10, discussed above, a controller 102, a computing device 104, and a feedback device 106. In some examples, the controller 102 and the computing device 104 are implemented on the same hardware device. The system 100 may also have an optional user interface 108. The controller 102 is operable to receive/collect temperature data from the sensors 14. The sensors 14 may include an integrated transmitter to send the temperature data to the controller 102, or may include a separate transmitter. Any transmission protocol known in the art could be used, including wireless transmission.

In one example, the controller 102 is operable to send a request to the sensors 14 to make a temperature measurement at a point in time upon receipt of an input from a user at the user interface 108. In another example, the controller 102 is operable to direct the sensors 14 to continually make temperature measurements at a predetermined time interval, such as one second.

The computing device 104 is configured to analyze temperature data collected by the controller 102. That is, the computing device 104 is programmed to analyze the temperature data to determine the cardiac/vascular health of the patient based on the temperature data from the sensors 14, as discussed above. The analysis can include machine learning and/or a neural network analysis. The analysis may include determining an absolute or relative indicator of blood flow in the patient's extremity, such as the patient's systemic vascular resistance (SVR) and cardiac output (CO). The analysis may also include, for instance, determining a change in the patient's cardiac/vascular profile over time. The results of the analysis may be displayed at the user interface 108.

The computing device 104 is also programmed to compare the actual cardiac/vascular profile of the patient as determined by the analysis discussed above with a predetermined “normal” cardiac/vascular profile of the patient. The “normal” status can be patient-specific and in some examples can be adjusted by a user at the user interface 108. The comparison can be in real-time or in near real-time, e.g., within seconds of the measurement being taken by the sensors 14.

If a deviation is detected by the computing device 104, the computing device 104 and/or the controller 102 are configured to cause the feedback device 106 to alert a user, such as a medical provider, of the deviation. The alert can be in real-time, or near real-time. The alert can be audible, visual, and/or tactile.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

1. A device for measuring a temperature profile of a patient's extremity, comprising:
a support structure having a plurality of openings therein;
a frame delineating each opening of the plurality of openings;
a sensor received in each frame; and
a plurality of rods connecting each sensor to each frame.

2. The device of claim 1, wherein at least one of the plurality of rods is adjustable in length.

3. The device of claim 2, wherein at least one of the plurality of rods includes a telescoping mechanism.

4. The device of claim 1, wherein the frame is rigid or semi-rigid.

5. The device of claim 4, wherein the frame is at least as rigid as the support structure.

6. The device of claim 1, wherein the support structure is flexible.

7. The device of claim 1, wherein the device includes at least 7 sensors.

8. A system for monitoring a temperature profile of a patient's extremity, comprising:
a device for measuring a temperature profile of a patient's extremity, the device comprising:
a support structure having a plurality of openings therein,
a frame delineating each opening of the plurality of openings,
a sensor received in each frame, and
a plurality of rods connecting each sensor to each frame;
a controller operable to receive temperature data from the sensors;
a computing device configured to analyze the temperature data from the sensors.

9. The system of claim 8, wherein the controller is configured to send a request to the sensors to make a temperature measurement.

10. The system of claim 8, wherein the computing device is configured to determine a cardiac profile of the patient based on analysis of the temperature data from the sensors.

11. The system of claim 10, wherein the computing device is configured to determine the patient's systemic vascular resistance based on analysis of the temperature data from the sensors.

12. The system of claim 10, wherein the computing device is configured to determine the patient's cardiac output based on analysis of the temperature data from the sensors.

13. The system of claim 8, wherein the computing device is configured to compare the cardiac profile of the patient to a predetermined normal cardiac profile.

14. The system of claim 8, wherein the computing device is configured to determine a change in the patient's cardiac profile over time based on analysis of the temperature data from the sensors.

15. The system of claim 8, further comprising a user interface configured to receive an input from a user and communicate the input to the controller.

16. (canceled)

17. (canceled)

18. (canceled)

19. (canceled)

20. (canceled)
